(19) United States
(12) Patent Application Publication
(10) Pub. No.: US 2025/0027936 A1
(43) Pub. Date: Jan. 23, 2025

(54) IMMUNOASSAY DEVICE, AND PREPARATION METHOD AND USE THEREOF

(71) Applicants: Peking University, Beijing (CN); PEKING UNIVERSITY FIRST HOSPITAL, Beijing (CN)

(72) Inventors: Yue CUI, Beijing (CN); Li YANG, Beijing (CN)

(73) Assignees: Peking University, Beijing (CN); PEKING UNIVERSITY FIRST HOSPITAL, Beijing (CN)

(21) Appl. No.: 18/407,977

(22) Filed: Jan. 9, 2024

(30) Foreign Application Priority Data
Jul. 17, 2023 (CN) 2023108755579

(51) Int. Cl. G01N 33/543; G01N 21/84

(52) U.S. Cl. CPC G01N 33/54387; G01N 21/8483; G01N 2470/04

(57) ABSTRACT

The present disclosure provides an immunoassay device, and a preparation method and use thereof, and belongs to the technical field of biological detection. In the present disclosure, the immunoassay device includes a test strip and a thin film pump, where a driving component in the thin film pump is connected to a printed circuit board (PCB) or a chip outside the thin film pump through a wire, to control starting or turning off of the thin film pump; and a liquid outlet of the thin film pump is closely attached to a top of a conjugate pad of the test strip, or a top of a sample pad of the test strip, or a top of a middle part between the conjugate pad and the sample pad.

### CROSS REFERENCE TO RELATED APPLICATION

This patent application claims the benefit and priority of Chinese Patent Application No. 2023108755579, filed with the China National Intellectual Property Administration on Jul. 17, 2023, the disclosure of which is incorporated by reference herein in its entirety as part of the present application.

### TECHNICAL FIELD

The present disclosure belongs to the technical field of biological detection, and in particular relates to an immunoassay device, and a preparation method and use thereof.

### BACKGROUND

Albumin is one of the most abundant and important proteins in the human circulatory system. As a member of the globular protein family, albumin is capable of binding and transporting various endogenous and exogenous compounds and plays an important role in human metabolism. With healthy kidneys, albumin in the blood does not pass into the urine, whereas if the kidneys are damaged, albumin does. Therefore, albumin in urine is an important biomarker of kidney diseases. The concentration of albumin in the urine of a healthy person usually does not exceed 30 mg/l, whereas the concentration of albumin in the urine of a patient is higher than 150 mg/L. Monitoring albumin in urine is important for monitoring disease progression in chronic kidney patients. At present, the traditional methods for detecting and quantifying albumin include classic colorimetric methods, such as sulfosalicylate test, Coomassie brilliant blue (Bradford) test, bromophenol blue test, and biuret reagent detection. However, these methods are inaccurate and not selective for the albumin in a mixture. More accurate instrumental techniques include visible absorption spectroscopy, electrochemical detection, immunofluorescence, enzyme-linked immunosorbent assay (ELISA), electrical or optical biosensors, and high-performance liquid chromatography (HPLC). However, these methods are generally high-cost, cumbersome, and time-consuming.

Lateral flow immunoassay (LFIA) has been widely used to detect diseases, pathogens, chemicals, toxins, and water pollutants. In recent years, the LFIA has been considered as one of the most promising point-of-care testing (POCT) tools in the field of bioanalysis, with obvious advantages such as simple operation, rapid analysis, low cost, portable equipment, and user-friendliness. Ki et al. simultaneously determined a total content of human serum albumin and a concentration of glycated albumin using colorimetric signals, with detection ranges of 1 ng/ml to 1 mg/mL and 0.5 μg/mL to 3.6 mg/mL, respectively. Zangheri developed a chemiluminescence-based LFIA integrated with an amorphous silicon (a-Si: H) photosensitive sensor array for the quantitative detection of albumin in urine with a limit of detection (LOD) of 2.5 mg/L. Yasukawa developed a test strip-based dual electrochemical sensor for the determination of albumin and creatinine, where the albumin shows a detection range of 18.75 μg/mL to 150 μg/mL. However, these detection ranges, or LODs, do not correspond to the concentration ranges in healthy individuals (with an albumin content of less than 30 mg/L) and kidney disease patients (with an albumin content of higher than 150 mg/L). Patients with kidney diseases tend to have a much higher albumin concentration, which presents a great challenge to the detection of LFIA. Excessively-high albumin concentrations limit rapid detection methods, making different concentrations in the LFIA indistinguishable. Urine samples need to be diluted in advance, but this process reduces the ease of use of LFIA. Therefore, it is a technical problem to be solved urgently at this stage to develop an LFIA device capable of detecting high-concentration albumin with a wide detection range and a high precision.

### SUMMARY

In view of this, an objective of the present disclosure is to provide an immunoassay device, and a preparation method and use thereof. The immunoassay device can directly conduct rapid and accurate quantitative detection of a nephropathy marker albumin in a high concentration range.

To achieve the above objective, the present disclosure provides the following technical solutions:

The present disclosure provides an immunoassay device, including a test strip and a thin film pump, where a liquid outlet of the thin film pump is closely attached to a top of a conjugate pad of the test strip, or a top of a sample pad of the test strip, or a top of a middle part between the conjugate pad and the sample pad.

Preferably, the thin film pump includes a driving component, a liquid inlet, the liquid outlet, and a solution storage chamber; and an electrode layer in the driving component is connected with a printed circuit board (PCB) or a chip outside the thin film pump through a wire.

Preferably, the thin film pump is selected from the group consisting of an electrochemical pump, an electroosmotic pump, and an ultrasonic pump.

Preferably, the conjugate pad of the test strip is coated with an antibody-conjugated optical marker; and the optical marker is selected from the group consisting of a fluorescent nanoparticle, a colloidal gold nanoparticle, and an enzyme that catalyzes chemiluminescence.

Preferably, an immunoassay method includes a competitive reaction or a sandwich immunoassay.

Preferably, in the competitive reaction, a nitrocellulose membrane of the test strip includes a test line and a control line, the test line is coated with a biomarker of a species to be tested, and the control line is coated with immunoglobulin G (IgG); and in the sandwich immunoassay, the nitrocellulose membrane of the test strip includes the test line and the control line, the test line is coated with an antibody of the biomarker of the species to be tested, and the control line is coated with the IgG.

The present disclosure further provides a preparation method of the immunoassay device, including the following steps:
connecting the electrode layer in the driving component in the thin film pump to the PCB or the chip outside the thin film pump through the wire, to control starting or turning off of the thin film pump: where the PCB or the chip is attached to a polyvinyl chloride (PVC) backing card on the other side of the test strip: fixing the nitrocellulose membrane on an upper surface of the PVC backing card, lap-jointing an absorbent pad to a tail end of the PVC backing card, and lap-jointing the sample pad and the conjugate pad sequentially to a head end of the PVC backing card to obtain the test strip; and attaching the liquid outlet of the thin film pump closely to the top of the conjugate pad of the test strip, or the top of the sample pad of the test strip, or the top of the middle part between the conjugate pad and the sample pad.

Preferably, the immunoassay device is placed in a 3D printed shell of a corresponding style; and the 3D printed shell is prepared by 3D printing or injection molding.

The present disclosure further provides use of an immunoassay device, including the following steps: adding a sample to the thin film pump of the immunoassay device, diluting the sample with a diluent in the thin film pump, starting the thin film pump to release a mixed solution of the sample and the diluent, such that the mixed solution flows to the conjugate pad and the nitrocellulose membrane in sequence, and observing or determining a color or an intensity of an optical signal on the test line and the control line in the immunoassay device: alternatively, adding the sample to the sample pad of the immunoassay device, starting the thin film pump to release the diluent, such that the sample and the diluent are mixed on the test strip to obtain the mixed solution and the mixed solution flows to the conjugate and the nitrocellulose membrane in sequence, and observing or determining the color or the intensity of the optical signal on the test line and the control line in the immunoassay device.

Preferably, the sample is diluted with the diluent by a factor of 1/10 to 1/1000000.

Compared with the prior art, the present disclosure has the following beneficial effects: In the present disclosure, a one-step fluorescent lateral flow quantitative analysis is conducted on human urine for the first time with a thin film pump, and a quantitative immunoassay device for detecting an albumin concentration in the urine is established by integrating a LFIA with a thin film pump. When a urine sample is added, the thin film pump first automatically releases a buffer to dilute the sample. An obtained diluted sample is then detected by a LFIA test strip, and a resulting determined fluorescence detection result is quantified.

In the present disclosure, the immunoassay device realizes one-step rapid and accurate detection of albumin, a marker of kidney diseases in a high concentration range, and can be directly used for the quantification of albumin in the urine of clinical patients with kidney diseases. The immunoassay device is helpful for hospitals or families to monitor the health of kidney disease patients, and also provides a comprehensive solution for the detection of high-concentration biomarkers in body fluids.

### BRIEF DESCRIPTION OF THE DRAWINGS

FIG. 1 shows a schematic diagram that the thin film pump is connected with the test strip in the immunoassay device;

FIG. 2 shows a 3D view of the shell;

FIG. 3 shows a schematic diagram of a detection principle of the immunoassay device;

FIGS. 4A-B show schematic view of the ultrasonic pump on the top of the sample pad of the test strip (where FIG. 4A is a front view, while FIG. 4B is a side view);

FIGS. 5A-B show schematic view of the ultrasonic pump on the top of the middle part between the sample pad and the conjugate pad of the test strip (where FIG. 5A is a front view, while FIG. 5B is a side view);

FIGS. 6A-B show schematic view of the ultrasonic pump on the top of the conjugate pad of the test strip (where FIG. 6A is a front view, while FIG. 6B is a side view);

FIGS. 7A-B show schematic view of the electrochemical pump on the top of the sample pad of the test strip (where FIG. 7A is a front view, while FIG. 7B is a side view);

FIGS. 8A-B show schematic view of the electrochemical pump on the top of the middle part between the sample pad and the conjugate pad of the test strip (where FIG. 8A is a front view, while FIG. 8B is a side view);

FIGS. 9A-B show schematic view of the electrochemical pump on the top of the conjugate pad of the test strip (where FIG. 9A is a front view, while FIG. 9B is a side view);

FIGS. 10A-B show schematic view of the electroosmotic pump on the top of the sample pad of the test strip (where FIG. 10A is a front view, while FIG. 10B is a side view);

FIGS. 11A-B show schematic view of the electroosmotic pump on the top of the middle part between the sample pad and the conjugate pad of the test strip (where FIG. 11A is a front view, while FIG. 11B is a side view);

FIGS. 12A-B show schematic view of the electroosmotic pump on the top of the conjugate pad of the test strip (where FIG. 12A is a front view, while FIG. 12B is a side view);

FIG. 13 shows screening results of the amount of antibody conjugated with fluorescent nanoparticles;

FIG. 14 shows screening results of the amount of antibody-conjugated fluorescent nanoparticles;

FIG. 15 shows the detection results of an albumin concentration on the test line;

FIG. 16 shows the detection results of sample addition locations;

FIG. 17 shows the detection results of sample dilution types;

FIG. 18 shows the detection results of dilutions of sample albumin concentrations;

FIG. 19 shows the detection results of the thin film pump at a position of the test strip;

FIG. 20 shows the detection results of a type of the thin film pump;

FIG. 21 shows the detection results of an addition position of antibody-conjugated fluorescent nanoparticles;

FIG. 22 shows an influence of presence or absence of the thin film pump on a detection performance of the immunoassay device;

FIG. 23 shows an influence of an albumin concentration in the sample on a flow rate of the thin film pump;

FIG. 24 shows a calibration curve for detecting albumin at an in-situ dilution of 1/1000;

FIG. 25 shows a calibration curve for albumin diluted at an in-situ dilution of 1/10000;

FIG. 26 shows the detection results of a detection time of the immunoassay device;

FIG. 27 shows the detection results of a specificity assay of the immunoassay device;

FIG. 28 shows the detection results of a stability assay of the immunoassay device;

FIG. 29 shows the quantitative analysis results of clinical samples; and

FIG. 30 shows the data and deviations between the concentrations of clinical samples determined by the immunoassay device and those determined by the immunoturbidimetric method used in the hospital.

Reference numerals: 1: thin film pump, 2: PCB or chip, 3: wire, 4: liquid inlet, 5: sample pad, 6: conjugate pad, 7: nitrocellulose membrane, 8: test line (T line), 9: control line (C line), 10: absorbent pad, 11: PVC backing card, 12: liquid outlet, 13: driving component, 14: solution storage chamber, 15: a shell area where the thin film pump is located, 16: a shell area where the test strip is located, and 17: visible area.

### DETAILED DESCRIPTION OF THE EMBODIMENTS

The present disclosure provides an immunoassay device, including a test strip and a thin film pump, where the thin film pump includes a driving component, a liquid inlet, a liquid outlet, and a solution storage chamber: an electrode layer in the driving component is connected to a PCB or a chip outside the thin film pump through a wire, to control starting or turning off of the thin film pump; and the liquid outlet of the thin film pump is closely attached to a top of a conjugate pad of the test strip, or a top of a sample pad of the test strip, or a top of a middle part between the conjugate pad and the sample pad. In the present disclosure, the immunoassay device is placed in a 3D printed shell of a corresponding style.

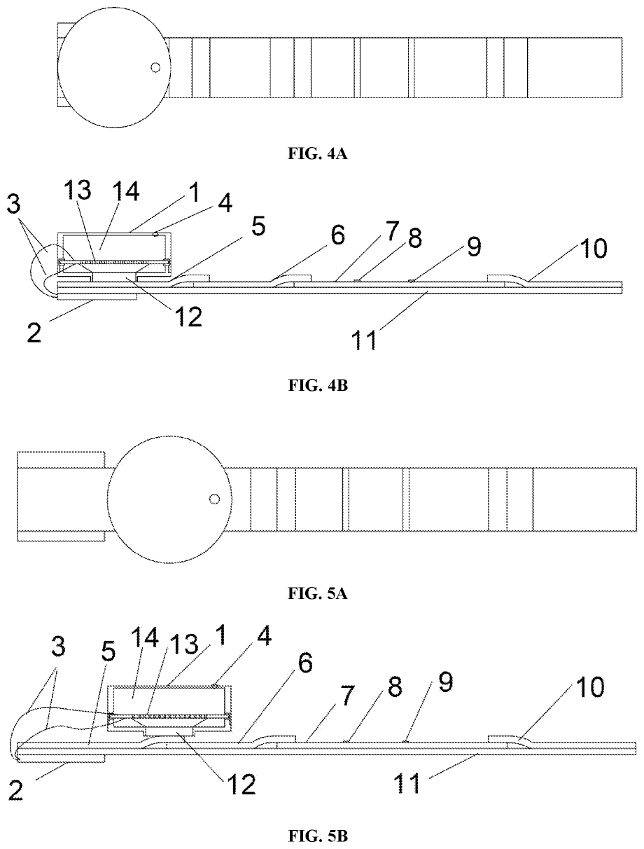

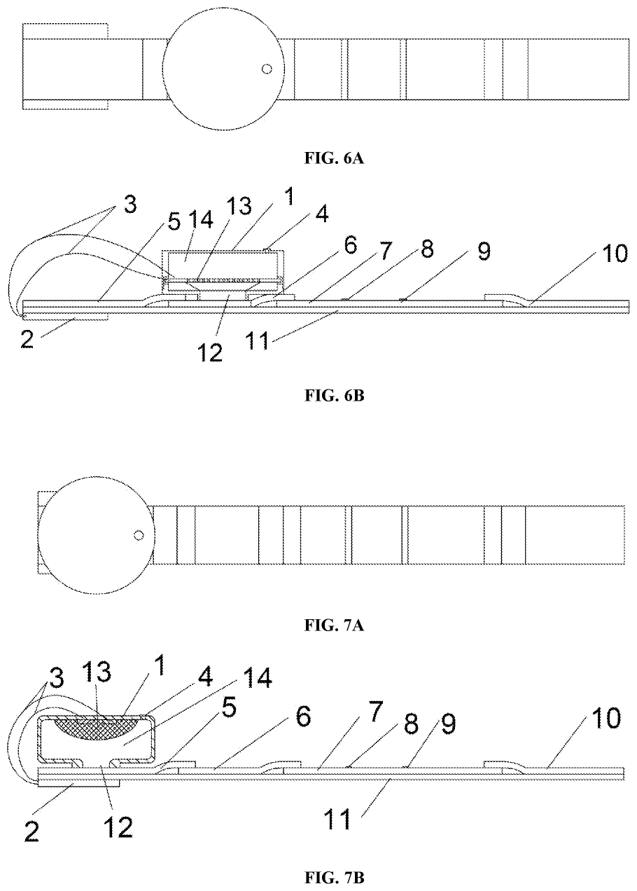

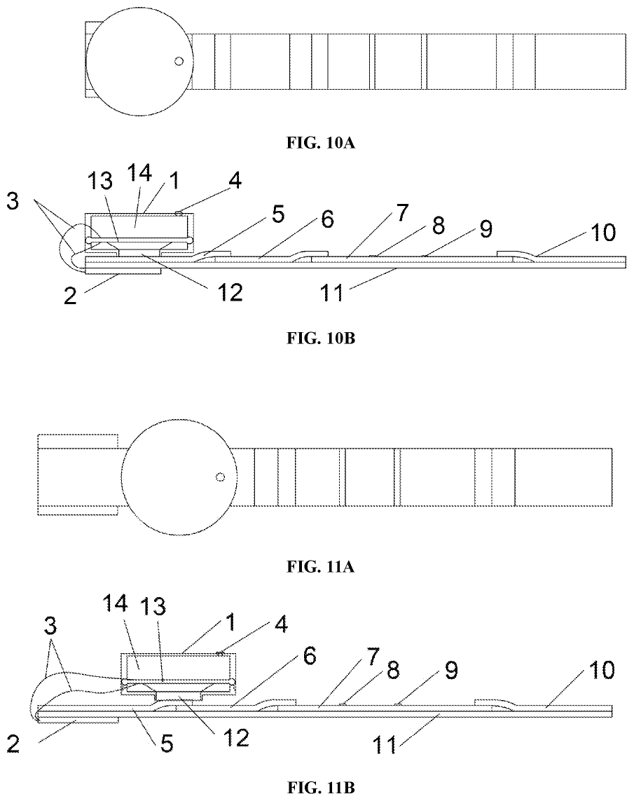

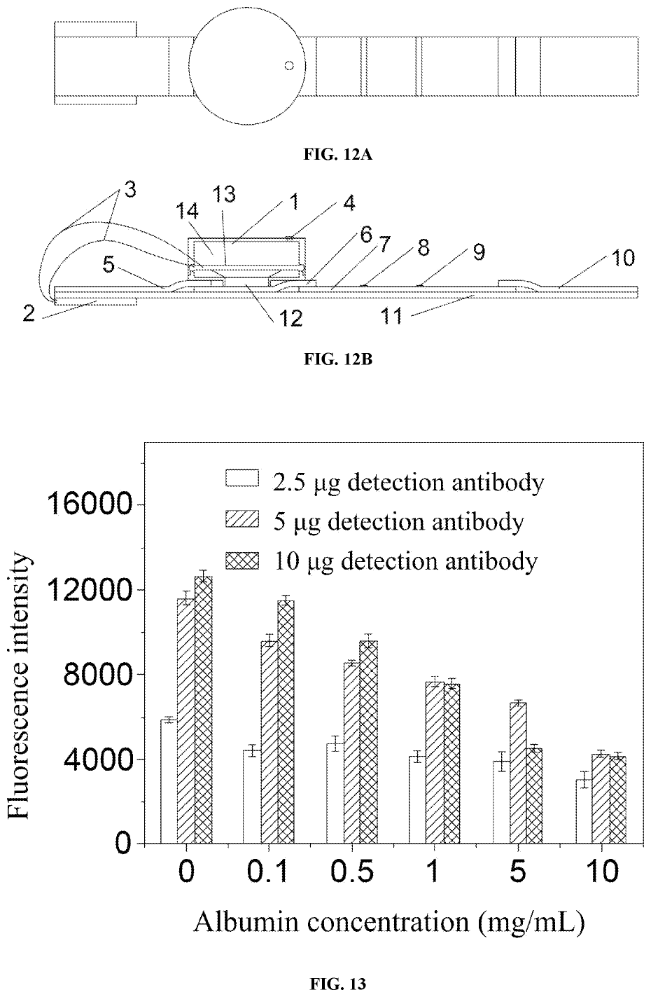

In the present disclosure, the thin film pump is selected from the group consisting of an ultrasonic pump, an electrochemical pump, and an electroosmotic pump. As a possible implementation, the ultrasonic pump (1 in FIGS. 4A-B to FIGS. 6A-B) is derived from a drug injection pump described in patent CN202211685429.X, where a membrane 30 and a piezoelectric ring 50 in the drug injection pump is the driving component in the present disclosure. As another possible implementation, the electrochemical pump (1 in FIGS. 7A-B to FIGS. 9A-B) refers to a drug injection pump in patent CN202210724624.2, where the drug injection pump as a drug storage component can be replaced with a cylindrical shape with a liquid outlet at the lower end, and a driving cavity in the driving component can be replaced with a hemispherical cavity. As a possible implementation, the driving component in the electroosmotic pump (1 in FIGS. 10A-B to FIGS. 12A-B) is derived from an electroosmotic pump in patent ZL202210393564.0; other structures in the electroosmotic pump except for the driving component (derived from ZL202210393564.0) are consistent with the ultrasonic pump in the present disclosure.

In the present disclosure, the thin film pump is used to store a diluent in advance, so as to dilute the sample. The electrode layer of the driving component in the thin film pump is connected to the PCB or chip outside the thin film pump through the wire, such that the PCB or chip can be controlled by the wire or wirelessly to control the starting or turning off of the thin film pump. The PCB or chip is integrated under the test strip and integrated with the immunoassay device, which can also be used as an external small hand-held instrument.

In the present disclosure, the test strip includes a sample pad, a conjugate pad, a nitrocellulose (NC) membrane, an absorbent pad, and a PVC backing card. The sample pad, the conjugate pad, the NC membrane, and the absorbent pad are sequentially overlapped and pasted on the backing card according to a tomography direction. The NC membrane is attached to a middle part of the PVC backing card, the absorbent pad is lap-jointed to a tail end of the PVC backing card, and the sample pad and the conjugate pad are lap-jointed to a head end of the PVC backing card: two ends of each component have an overlap of 1.5 mm to 2.5 mm to ensure continuous flow of an antibody or antigen from the sample pad to the absorbent pad under capillary action. The sample pad and the absorbent pad can be purchased from commercial sources.

In the present disclosure, the conjugate pad of the test strip is coated with an antibody-conjugated optical marker; and the optical marker is selected from the group consisting of a fluorescent nanoparticle, a colloidal gold nanoparticle, and an enzyme that catalyzes chemiluminescence. The antibody-conjugated optical marker is preferably antibody-conjugated fluorescent nanoparticles, with an amount of 1 μL to 5 μL, preferably 1.5 μL to 4.5 μL; and the antibody-conjugated fluorescent nanoparticles are albumin antibody-conjugated fluorescent nanoparticles. During preparation of the albumin antibody-conjugated fluorescent nanoparticles, an albumin antibody is added at 4 μg to 15 μg, preferably 5 μg to 10 μg. The antibody-conjugated fluorescent nanoparticles are obtained by conjugating the fluorescent nanoparticles with the antibody through an EDC-NHS chemical bond method.

In the present disclosure, an immunoassay method includes a competitive reaction or a sandwich immunoassay. In the competitive reaction, the NC membrane of the test strip includes a test line (T line) and a control line (C line): the test line is coated with a biomarker of a species to be tested, and the control line is coated with IgG: preferably, the test line is coated with albumin and the control line is coated with goat anti-mouse IgG. The concentration of coated albumin is 0.5-1.8 mg/mL, preferably 1.5 mg/mL; and the concentration of goat anti-mouse IgG is 0.5-1.8 mg/mL, preferably 1 mg/mL. Albumin and goat anti-mouse IgG are dispensed sequentially on the test line and the control line of the NC membrane at an injection speed of (0.5-1.5) μL/cm, respectively. The test line and the control line are separated by 2 mm to 4 mm. The NC membrane coated with albumin and goat anti-mouse IgG is dried at room temperature for 0.8 h to 1.5 h, then cut into membrane strips with a width of 4 mm to 6 mm, and then stored in a sealed bag with a desiccant for later use.

In the sandwich immunoassay of the present disclosure, the NC membrane of the test strip includes the test line and the control line: the test line is coated with an antibody of the biomarker of the species to be tested, and the control line is coated with IgG. The antibody of the biomarker of the species to be tested is preferably the albumin antibody, and IgG is preferably the goat anti-mouse IgG.

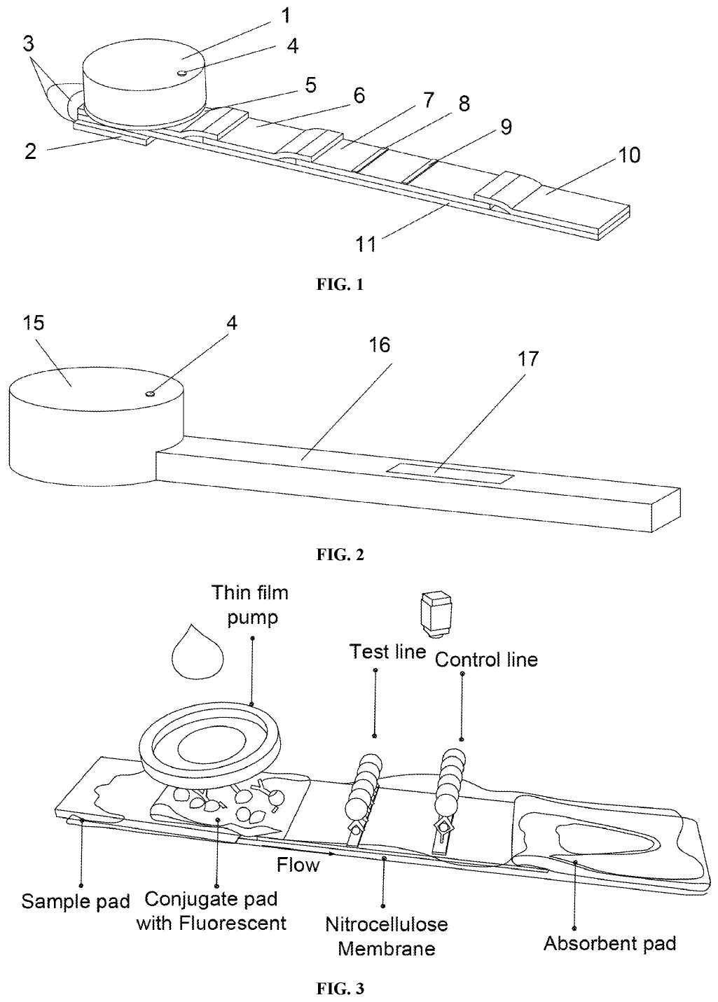

The present disclosure further provides a preparation method of the immunoassay device, including the following steps:
connecting the electrode layer in the driving component in the thin film pump to the PCB or the chip outside the thin film pump through the wire, to control starting or turning off of the thin film pump: where the PCB or the chip is attached to a PVC backing card on the other side of the test strip: fixing the NC membrane on an upper surface of the PVC backing card, lap-jointing an absorbent pad to a tail end of the PVC backing card, and lap-jointing the sample pad and the conjugate pad sequentially to a head end of the PVC backing card to obtain the test strip; and attaching the liquid outlet of the thin film pump closely to the top of the conjugate pad of the test strip, or the top of the sample pad of the test strip, or the top of the middle part between the conjugate pad and the sample pad. In the present disclosure, the immunoassay device is placed in a 3D printed shell of a corresponding style; and the 3D printed shell is prepared by 3D printing or injection molding. The PCB or chip can be adhered to the PVC backing card on an opposite side of the sample pad and at the head end of the test strip, as shown in FIG. 1. The 3D printed shell is shown in FIG. 2, where the shell shown in this figure is a shell used after the thin film pump is assembled above the sample pad. Alternatively, according to an assembled style that the thin film pump is placed on the top of the middle part between the sample pad and the conjugate pad, or an assembled style that the thin film pump is placed on the top of the conjugate pad, the 3D printed shell of the corresponding style is obtained by 3D printing or injection molding. The PCB or chip can control the starting or turning off of the thin film pump through a wired or wireless receiver.

The present disclosure further provides use of an immunoassay device, including the following steps: adding a sample to the thin film pump of the immunoassay device, diluting the sample with a diluent in the thin film pump, starting the thin film pump to release a mixed solution of the sample and the diluent, such that the mixed solution flows to the conjugate pad and the NC membrane in sequence, and observing or determining a color or an intensity of an optical signal (including particle color, fluorescence, and chemiluminescence) on the test line and the control line in the immunoassay device: alternatively: adding the sample to the sample pad of the immunoassay device, starting the thin film pump to release the diluent, such that the sample and the diluent are mixed on the test strip to obtain the mixed solution and the mixed solution flows to the conjugate and the NC membrane in sequence, and observing or determining the color or the intensity of the optical signal (including particle color, fluorescence, and chemiluminescence) on the test line and the control line in the immunoassay device. The diluent is a PBS solution (10 mM, pH=7.4), which can be prepared according to conventional methods or purchased from commercial sources. The sample is diluted with the diluent by a factor of 1/1000 to 1/10000.

In the present disclosure, a process of detecting albumin in urine with the immunoassay device includes (FIG. 3): a sample containing albumin is placed on the thin film pump or the sample pad of the immunoassay device, and when the sample flows to the conjugate pad, the antibody-conjugated fluorescent nanoparticles bind to albumin in the sample and continue to flow along the NC membrane. Albumin on the test line of the NC membrane cannot bind to the albumin antibody-conjugated fluorescent nanoparticles because binding sites are occupied. The antibody on the control line of the NC membrane binds to the albumin antibody-conjugated fluorescent nanoparticles. 365 nm ultraviolet light is used as an excitation light, the fluorescent nanoparticles on the control line emit red fluorescence. When there is albumin in the sample, there is no fluorescent signal on the test line. When there is no albumin in the sample, the fluorescent nanoparticles on both the test line and the control line emit red fluorescence.

The technical solution provided by the present disclosure will be described in detail below with reference to the examples, but they should not be construed as limiting the claimed scope of the present disclosure.

### Example 1 Preparation of Immunoassay Device

1. Preparation of Antibody-Conjugated Fluorescent Nanoparticles: Fluorescent Nanoparticles were Conjugated with an Antibody Through an EDC-NHS Chemical Bond Method.

1 mL of an activation buffer (20 mM MES, pH=6.5) was added to 200 μL of a fluorescent nanoparticle solution to activate carboxyl groups on a surface of the fluorescent nanoparticles. A resulting mixed solution was centrifuged at 13,000 rpm/min for 20 min, a supernatant was removed, and the fluorescent nanoparticles were resuspended in 200 μL of the activation buffer. 5 mg of freshly prepared EDC (1% EDC in the activation buffer) was added to a resulting mixed solution and mixed to allow reaction for 5 min.

15 mg of NHS (5% NHS dissolved in activation buffer) was added, and mixed to allow reaction for 10 min. At this time, the carboxyl groups on the surface of the fluorescent nanoparticles were activated. The mixed solution was washed three times with activation buffer, centrifuged at 13,000 r/min for 20 min, and a supernatant was separated after centrifugation, and the fluorescent nanoparticles were resuspended in 400 μL of a coupling buffer (20 mM MES, pH=8.5). 5 μg of an antibody was added to a resulting mixed solution, mixed well on a vortex suspension, and reacted at room temperature for 2 h. 100 μL of a blocking buffer (10% BSA+water) was added to an obtained binding solution, mixed well, and reacted at room temperature for 30 min, such that other reaction sites of the fluorescent nanoparticles were blocked to avoid non-specific reactions. A resulting mixed solution was centrifuged at 13,000 r/min for 10 min, a supernatant was separated, and the fluorescent nanoparticles were washed three times with the coupling buffer. The nanoparticle-conjugated antibody was suspended in 200 μL of a storage solution (including 1% BSA, 0.05% Tween-20, 0.05% sucrose, and 20 mM BBS buffer, pH=8.5), and stored in a refrigerator at 4° C.

### 2. Preparation of Immunoassay Device

### (1) Assembly of a Test Strip

Materials: a sample pad (5×13.5 mm²), a conjugate pad (5×6.5 mm²), an NC membrane (5×25 mm²), an absorbent pad (5×15 mm²), and a PVC backing card.

Preparation of the conjugate pad: 2.5 μL of the antibody-conjugated fluorescent nanoparticles prepared in step 1 were added dropwise on the conjugate pad, and then dried for later use.

Preparation of the NC membrane: the NC membrane was attached to a middle part of the PVC backing card: 1.5 mg/mL albumin and 1 mg/mL goat anti-mouse IgG were dispensed on the NC membrane through an XYZ platform dispenser at an injection speed of 1 μL/cm: where a line dispensed with albumin was a test line, and a line dispensed with goat anti-mouse IgG was a control line, and the two lines were separated by 3 mm: the dispensed NC membrane was dried at room temperature for 1 h, cut into 5 mm wide membrane strips, and then stored in a sealed bag with a desiccant for future use.

Assembly of the test strip: the prepared PVC backing card connected to the NC membrane was taken, the absorbent pad was connected to a tail end of the PVC backing card, while the sample pad and the conjugate pad were connected to a head end of the backing card. The two ends of each component had an overlap of 2 mm to ensure continuous flow of the antibody from the sample pad to the absorbent pad under capillary action.

### (2) Assembly of the Immunoassay Device

In an ultrasonic pump (a drug injection pump derived from patent CN115887815A), edges on opposite sides of a thin film and a piezoelectric ring were separately connected to a PCB or chip outside the ultrasonic pump through wires, to control starting or turning off of the ultrasonic pump:
the PCB or chip was adhered to the PVC backing card on an opposite side of the sample pad and at the head end of the test strip;
a liquid outlet of the ultrasonic pump was closely attached to a top of the sample pad of the test strip (FIGS. 4A-B), or a top of a middle part between the sample pad and the conjugate pad (FIGS. 5A-B), or a top of the conjugate pad of the test strip (FIGS. 6A-B);
a 3D printed shell was prepared by 3D printing or injection molding according to the styles of the above three assembled immunoassay devices; and
the immunoassay device was put into the 3D printed shell.

### Example 2 Preparation of Immunoassay Device

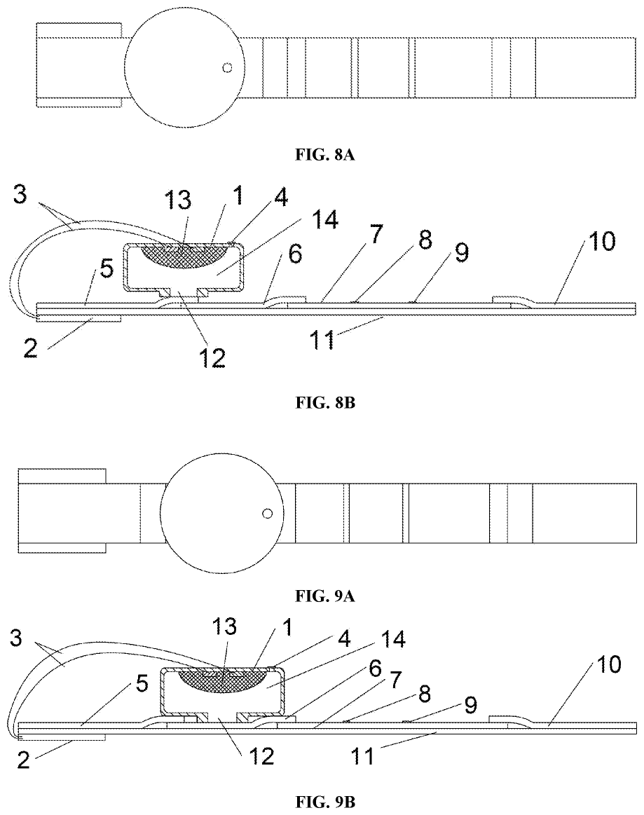

This example differed from Example 1 in that: the ultrasonic pump was replaced by an electrochemical pump, and a liquid outlet of the electrochemical pump was closely attached to a top of the sample pad of the test strip (FIGS. 7A-B), or a top of a middle part between the sample pad and the conjugate pad (FIGS. 8A-B), or a top of the conjugate pad of the test strip (FIGS. 9A-B); while other steps were the same as those in Example 1.

The electrochemical pump referred to a drug injection pump in patent CN202210724624.2, where the drug injection pump as a drug storage component could be replaced with a cylindrical shape with a liquid outlet at the lower end, as shown in 14 of FIGS. 7A-B to FIGS. 9A-B, and a driving cavity in the driving component could be replaced with a hemispherical cavity, as shown in 13 of FIGS. 7A-B to FIGS. 9A-B.

### Example 3 Preparation of Immunoassay Device

This example differed from Example 1 in that: the ultrasonic pump was replaced by an electroosmotic pump, and a liquid outlet of the electroosmotic pump was closely attached to a top of the sample pad of the test strip (FIGS. 10A-B), or a top of a middle part between the sample pad and the conjugate pad (FIGS. 11A-B), or a top of the conjugate pad of the test strip (FIGS. 12A-B): while other steps were the same as those in Example 1.

The driving component in the electroosmotic pump (1 in FIGS. 10A-B to FIGS. 12A-B) was derived from an electroosmotic pump in patent ZL202210393564.0; other structures in the electroosmotic pump except for the driving component (the patent above) were consistent with the ultrasonic pump in the present disclosure.

### Example 4 Detection Method of Immunoassay Device

0.1 μL of a sample was added dropwise to the thin film pump of the immunoassay device, and diluted with 999.9 μL of a PBS solution (10 mM, pH=7.4) contained in the thin film pump. A mixture of the sample and the PBS solution was released by electrically operating the thin film pump, and the mixture flowed to the conjugate pad and the NC membrane in sequence. After 5 min, a fluorescent signal on the test line was detected by an immunoassay analyzer under the light of 365 nm, which excited the beads to generate the fluorescent signal. The immunoassay analyzer could determine a fluorescent signal intensity of the test line.

### Example 5 Detection Method of Immunoassay Device

This example differed from Example 4 in that: the sample was added dropwise onto the sample pad at a volume of 0.1 μL, and the thin film pump contained 999.9 μL of PBS solution; electric operation started the thin film pump to release the PBS solution: after the sample was mixed with the PBS solution on the test strip, a resulting mixture flowed to the conjugate pad and the NC membrane in sequence. Other steps were the same as those in Example 4.

### Example 6 Preparation and Detection Optimization of Immunoassay Device

1. Screening of the Amount of Antibody Conjugated with Fluorescent Nanoparticles

The amount of antibody conjugated with fluorescent nanoparticles was set at 2.5 μg, 5 μg, and 10 μg, respectively; antibody-conjugated fluorescent nanoparticles with the above three antibody amounts were prepared by the preparation method of antibody-conjugated fluorescent nanoparticles in step 1 of Example 1, where the antibody was albumin antibody. The immunoassay device was prepared by the preparation method of the immunoassay device in step 2 of Example 1, as shown in FIGS. 5A-B.

The concentration of albumin in the samples was set as 0, 0.1 mg/mL, 0.5 mg/mL, 1 mg/mL, 5 mg/mL, and 10 mg/mL, respectively. The samples with different albumin concentrations were determined by the detection method in Example 4, and the immunoassay analyzer could determine the fluorescent signal intensity of the test line. The results were shown in FIG. 13.

As shown in FIG. 13, different antibody amounts (2.5 μg, 5 μg, and 10 μg) conjugated to fluorescent nanoparticles had an effect on the color intensity and sensitivity of LFIA. 5 μg and 10 μg of antibodies conjugated to the fluorescent nanoparticles had better detection strength and sensitivity. There was not much difference in test results between 5 μg and 10 μg of antibodies, which may be due to the saturation of antibody on the surface of fluorescent nanoparticles. Therefore, 5 μg of antibody was selected to be conjugated with nanoparticles to prepare antibody-conjugated fluorescent nanoparticles, which were used to prepare the immunoassay device.

### 2. Screening of the Amount of Antibody-Conjugated Fluorescent Nanoparticles

The antibody-conjugated fluorescent nanoparticles were prepared by the method in step 1 of Example 1: the antibody-conjugated fluorescent nanoparticles were set at 1 μL, 2.5 μL, and 5 μL, where the antibody was an albumin antibody: the immunoassay device was prepared by the preparation method of the immunoassay device in step 2 of Example 1, as shown in FIGS. 5A-B.

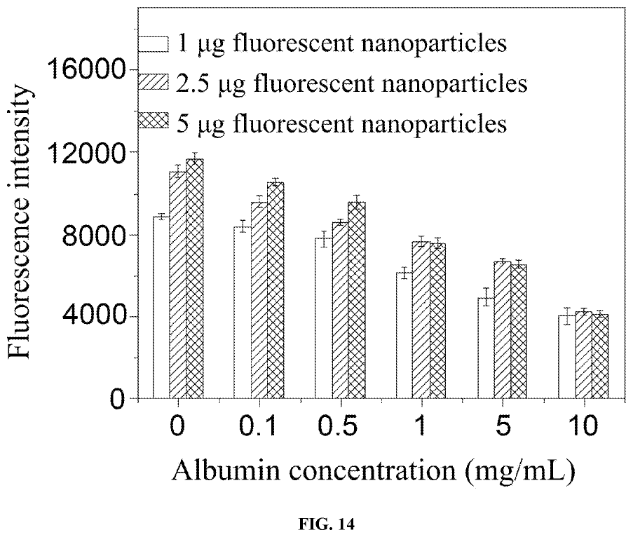

The concentration of albumin in the samples was set as 0, 0.1 mg/mL, 0.5 mg/mL, 1 mg/mL, 5 mg/mL, and 10 mg/mL, respectively. The samples with different albumin concentrations were determined by the detection method in Example 4, and the immunoassay analyzer could determine the fluorescent signal intensity of the test line. The results were shown in FIG. 14.

As shown in FIG. 14, 1 μL of the antibody-conjugated fluorescent nanoparticles resulted in reduced signal intensity, while 2.5 μL and 5 μL of the antibody-conjugated fluorescent nanoparticles had better fluorescence intensity and sensitivity. At the same time, compared with 2.5 μL of the antibody-conjugated fluorescent nanoparticles, the signal intensity of the test results of the immunoassay device prepared with 5 μL of the antibody-conjugated fluorescent nanoparticles did not improve much, such that 2.5 μL of fluorescent nanoparticles was selected to be coated in the conjugate pad to prepare the immunoassay device.

### 3. Screening of a Concentration of Capture Antibody (Albumin Antibody) on the Test Line

The antibody-conjugated fluorescent nanoparticles were prepared by the method in step 1 of Example 1, where the antibody was albumin antibody. The albumin antibody concentration on the test line was set to 0.5 mg/mL, 1 mg/mL. 1.5 mg/mL, 2 mg/mL separately: the immunoassay device was prepared by the preparation method of the immunoassay device in step 2 of Example 1, as shown in FIGS. 5A-B.

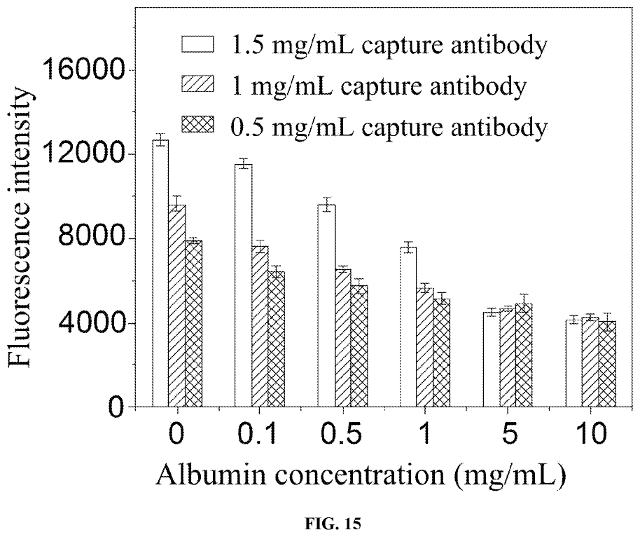

The concentration of albumin in the samples was set as 0, 0.1 mg/mL, 0.5 mg/mL, 1 mg/mL, 5 mg/mL, and 10 mg/mL, respectively. The samples with different albumin concentrations were determined by the detection method in Example 4, and the immunoassay analyzer could determine the fluorescent signal intensity of the test line. The results were shown in FIG. 15.

As shown in FIG. 15, the signal intensity increased as the albumin antibody concentration of the test line increased. When the albumin antibody concentration of the test line exceeded 2 mg/mL, a false positive test result occurred, which was a false signal and not displayed. Therefore, 1.5 mg/mL was the most suitable albumin concentration for the test line.

### 4. Screening of Sample Addition Locations

The antibody-conjugated fluorescent nanoparticles were prepared by the method in step 1 of Example 1, where the antibody was albumin antibody. The immunoassay device was prepared by the preparation method of the immunoassay device in step 2 of Example 1, as shown in FIGS. 5A-B.

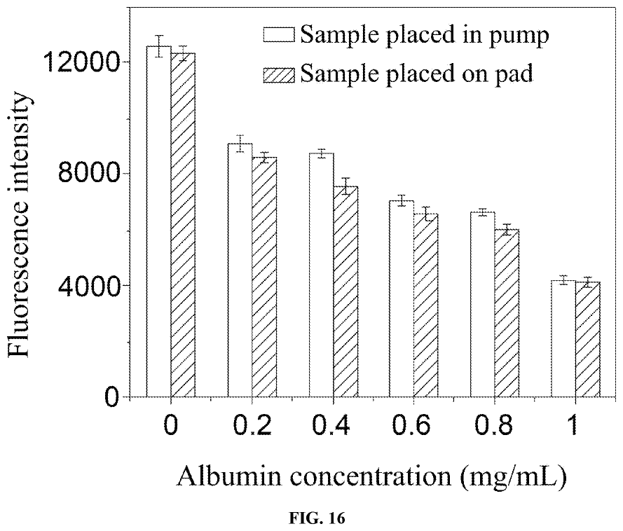

The concentration of albumin in the sample was separately set to 0, 0.2 μg/mL, 0.4 μg/mL, 0.6 μg/mL, 0.8 μg/mL, and 1 μg/mL, the samples with different albumin concentrations were determined by the detection methods in Examples 4 and 5, and the immunoassay analyzer could determine the fluorescent signal intensity of the test line. The results were shown in FIG. 16.

As shown in FIG. 16, there was no difference in test results when the sample was added to the thin film pump or to the sample pad. Due to the small amount of sample added, whether it was added to the sample pad or the pump, the sample could be diluted quickly. There was no difference in test results.

### 5. Screening of Sample Dilution Types

The antibody-conjugated fluorescent nanoparticles were prepared by the method in step 1 of Example 1, where the antibody was albumin antibody. The immunoassay device was prepared by the preparation method of the immunoassay device in step 2 of Example 1, as shown in FIGS. 5A-B.

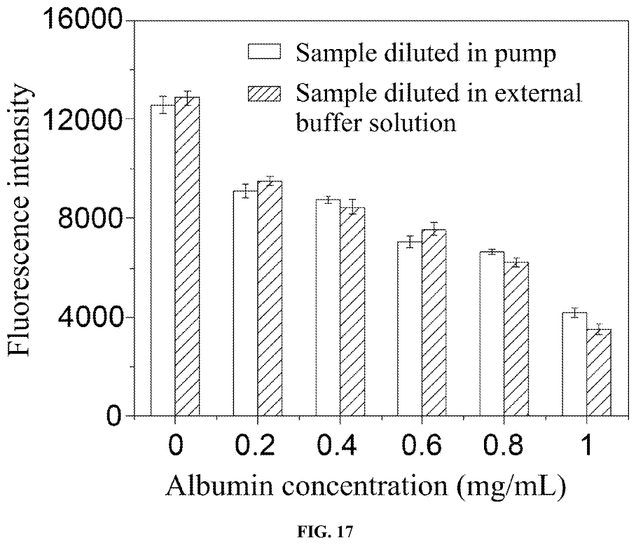

The concentration of albumin in the sample was set to 0, 0.2 μg/mL, 0.4 μg/mL, 0.6 μg/mL, 0.8 μg/mL, and 1 μg/mL, respectively, the samples at different albumin concentrations were diluted in the thin film pump or in an external PBS solution, the samples with different albumin concentrations were determined by the detection method in Examples 4, and the immunoassay analyzer could determine the fluorescent signal intensity of the test line. The results were shown in FIG. 17.

As shown in FIG. 17, there was a high consistency regardless of whether the sample was diluted in the thin film pump or in the external PBS solution. This shows that the thin film pump had a desirable dilution effect, and could simplify the dilution steps to achieve a one-step detection.

### 6. Screening of Dilutions of Sample Albumin Concentrations

The antibody-conjugated fluorescent nanoparticles were prepared by the method in step 1 of Example 1, where the antibody was albumin antibody. The immunoassay device was prepared by the preparation method of the immunoassay device in step 2 of Example 1, as shown in FIGS. 5A-B.

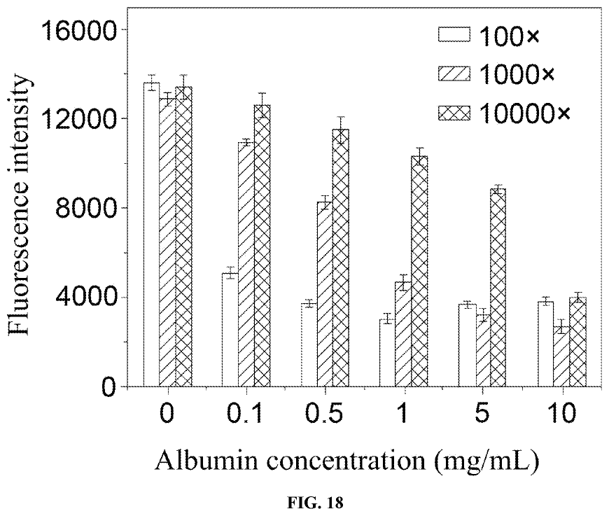

The concentration of albumin in the sample was set to 0, 0.1 mg/mL, 0.5 mg/mL, 1 mg/mL, 5 mg/mL, and 10 mg/mL respectively, different amounts of PBS solutions were stored in the thin film pump, and the sample albumin concentrations were diluted to 1/100, 1/1000, and 1/10000 of the original. The samples with different albumin concentrations were determined by the detection method in Example 4, and the immunoassay analyzer could determine the fluorescent signal intensity of the test line. The results were shown in FIG. 18.

As shown in FIG. 18, when the albumin concentration was diluted to 1/1000 of the original, it was difficult to distinguish between albumin concentrations higher than 1 mg/mL in the test results. When the albumin concentration was diluted to 1/10000, there was the strongest signal intensity, and the test result had also the widest linear range. When the albumin concentration was diluted to 1/100, different concentrations of albumin could not be distinguished in the test results. In order to obtain a stronger signal, the albumin concentration was diluted to 1/10000 of the original.

### 7. Screening of the Thin Film Pump at a Position of the Test Strip

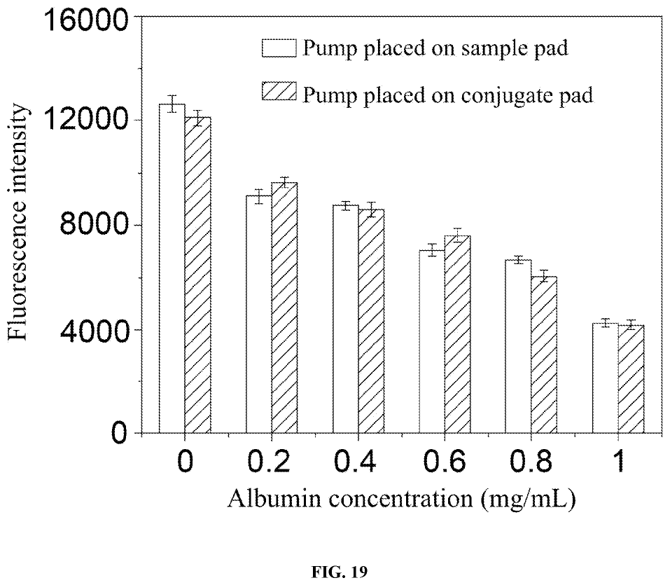

The concentration of albumin in the sample was set to 0, 0.2 μg/mL, 0.4 μg/mL, 0.6 μg/mL, 0.8 μg/mL, and 1 μg/mL, respectively, the immunoassay device prepared in Example 1 was used, as shown in FIGS. 4A-B and FIGS. 6A-B, the samples with different albumin concentrations were determined by the detection method in Example 4, and the immunoassay analyzer could determine the fluorescent signal intensity of the test line. The results were shown in FIG. 19.

As shown in FIG. 19, the position of the thin film pump on the test strip had no influence on the test results. The pump was located not far apart, thus not affecting the dilution and mixing of the liquids, resulting in similar test results.

### 8. Screening of a Type of the Thin Film Pump

The immunoassay device prepared in Example 1 was used, and the thin film pump was set as an ultrasonic pump or an electroosmotic pump, as shown in FIGS. 5A-B and FIG. 11A-B.

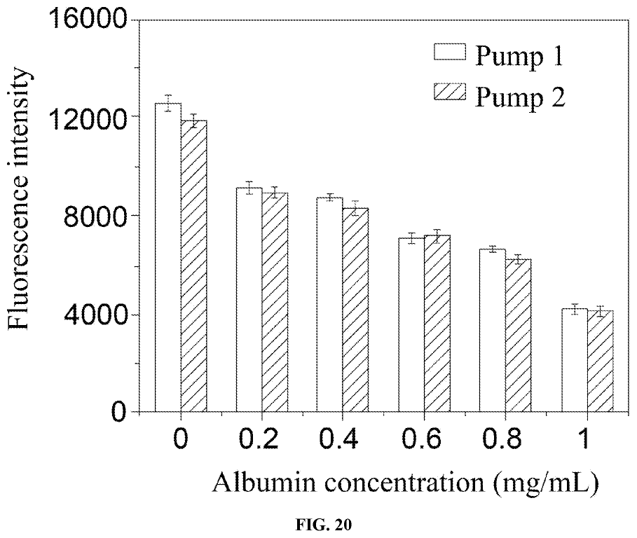

The concentration of albumin in the sample was set to 0, 0.2 μg/mL, 0.4 μg/mL, 0.6 μg/mL, 0.8 μg/mL, and 1 μg/mL, respectively, the samples with different albumin concentrations were determined by the detection method in Example 4, and the immunoassay analyzer could determine the fluorescent signal intensity of the test line. The results were shown in FIG. 20.

As shown in FIG. 20, the type of the thin film pump had no influence on the test results. The pump was located not far apart, thus not affecting the dilution and mixing of the liquids, resulting in similar test results.

### 9. Screening of an Addition Position of Antibody-Conjugated Fluorescent Nanoparticles

The immunoassay device was prepared in Example 1, as well as the immunoassay device was prepared by placing the antibody-conjugated fluorescent nanoparticles in the thin film pump according to the preparation method in Example 1, as shown in FIGS. 5A-B.

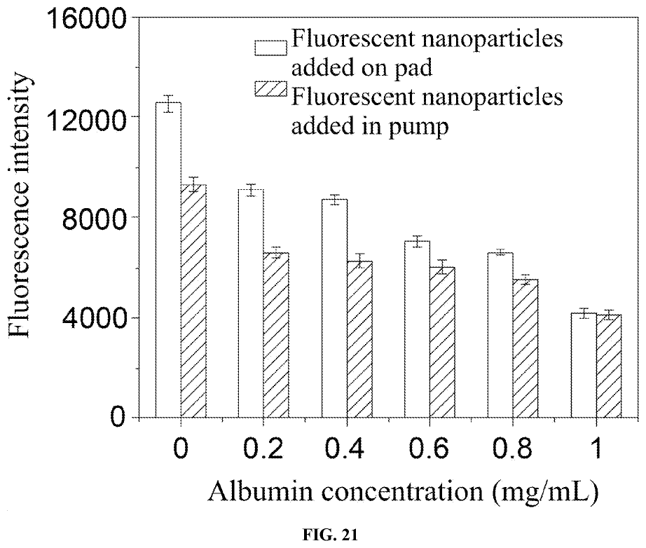

The concentration of albumin in the sample was set to 0, 0.2 μg/mL, 0.4 μg/mL, 0.6 μg/mL, 0.8 μg/mL, and 1 μg/mL, respectively, the samples with different albumin concentrations were determined by the detection method in Example 4, and the immunoassay analyzer could determine the fluorescent signal intensity of the test line. The results were shown in FIG. 21.

As shown in FIG. 21, two different methods could achieve the detection of albumin, but the method of antibody-conjugated fluorescent nanoparticles on the conjugate pad made the detection results more accurate, and the linear range of detection was wider. This was because the flow of antibody-conjugated fluorescent nanoparticles was affected by the thin film pump, and some antibody-conjugated fluorescent nanoparticles remained in the thin film pump, resulting in weak signal intensity.

### Example 7 Performance Detection of Immunoassay Device

1. Influence of Presence or Absence of the Thin Film Pump on a Detection Performance of the immunoassay device

The antibody-conjugated fluorescent nanoparticles were prepared by the method in step 1 of Example 1, where the antibody was albumin antibody. The immunoassay device with the thin film pump and the immunoassay device without the thin film pump were prepared according to the preparation method of the immunoassay device in step 2 of Example 1, where the immunoassay device with the thin film pump was shown in FIGS. 5A-B.

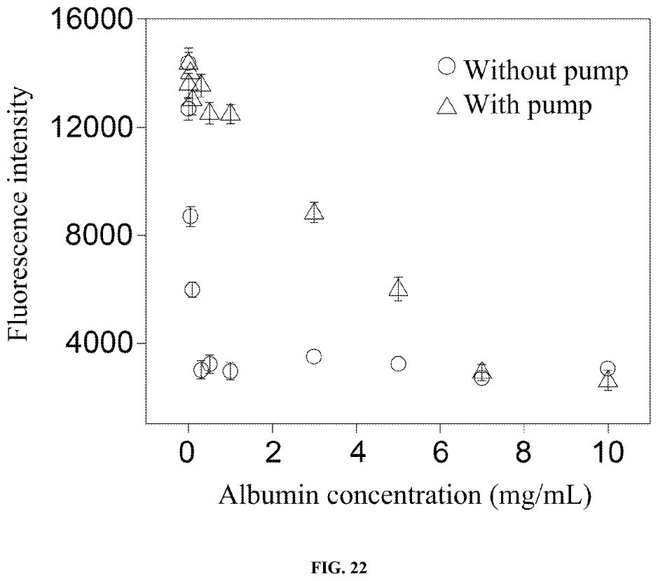

The samples with the albumin concentrations of 0-10 mg/mL were determined by detection method in Example 4, and the immunoassay analyzer could determine the fluorescent signal intensity of the test line. The results were shown in FIG. 22.

As shown in FIG. 22, when the albumin concentration in the sample was higher than 1 mg/mL, the fluorescent intensity on the test line of the immunoassay device without the thin film pump did not change much, and quantitative detection could not be realized. The albumin samples of different concentrations ((0-10) mg/mL) could be differentiated by the immunoassay device with the thin film pump. This showed that the thin film pump in the immunoassay device could effectively improve the detection range.

### 2. Influence of an Albumin Concentration in the Sample on a Flow Rate of the Thin Film Pump

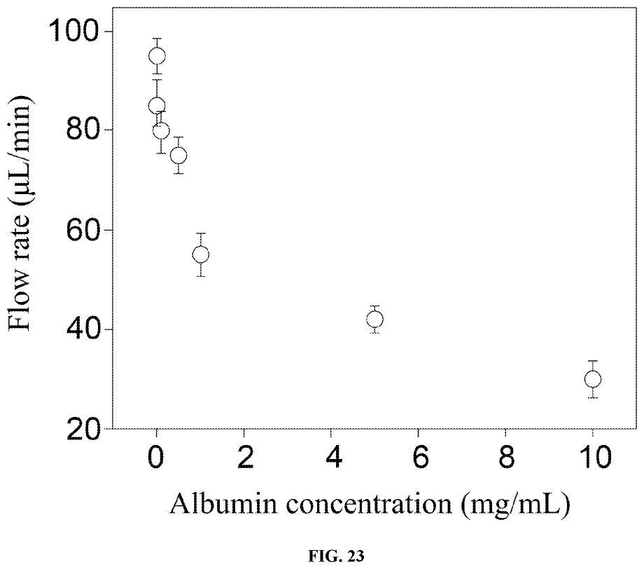

The albumin concentration of the sample was set to 0, 10 μg/ml, 100 μg/ml, 500 μg/ml, 1 mg/ml, 5 mg/ml, and 10 mg/ml, respectively; the immunoassay device prepared in Example 1 was used, as shown in FIGS. 5A-B: the samples with different albumin concentration were determined by the detection method of Example 4: a time required for the solution to be released from the thin film pump was recorded, and the corresponding flow rate for each albumin sample was calculated. The results were shown in FIG. 23.

As shown in FIG. 23, the flow rate of the thin film pump varied with the concentration of albumin in the sample. As the concentration of albumin increased, the flow rate decreased. When the albumin concentration in the thin film pump was 0.1 mg/ml, the flow rate of the thin film pump could reach 100 μL/min. When the albumin concentration was 10 mg/mL, the flow rate was decreased to 10 μL/min. In the assay, 999.9 μL of buffer solution was added to the thin film pump, and then 0.1 μL of sample was added dropwise into the pump. Since the concentration was diluted to 1/10000, the detection could be achieved at high flow rates.

### 3. Test of a Calibration Curve of the Albumin Sample Diluted to 1/1000 and 1/10000

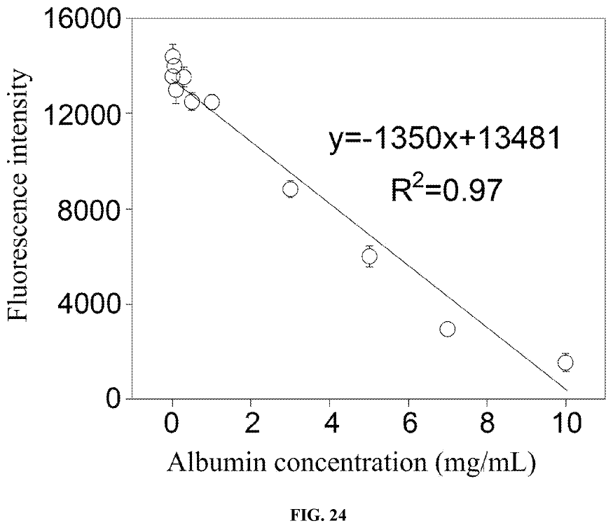

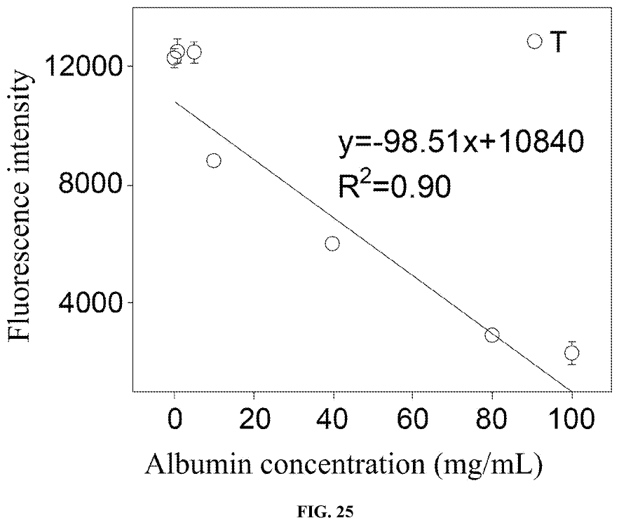

The immunoassay device prepared in Example 1 was used, as shown in FIGS. 5A-B. The detection method in Example 4 was used, where the thin film pump contained 99.9 μL of PBS solution or 999.9 μL of PBS solution, the sample was added dropwise to the thin film pump at a volume of 0.1 μL, and the samples with the protein concentrations of 0-10 mg/mL or 0-100 mg/mL were tested, respectively. The immunoassay analyzer could determine a fluorescent intensity on the test line. The results were shown in FIG. 24 to FIG. 25.

As shown in FIG. 24, when the albumin was diluted to 1/1000, a linear range of the calibration curve was 0-10 mg/mL, and a linear equation of the added albumin sample was y=−1350x+13481 (x was albumin concentration, y was a test value T), with a coefficient R² of 0.97.

As shown in FIG. 25, when the albumin sample was diluted to 1/10000, a linear range of 0-100 mg/mL could be achieved. A linear equation of the albumin detection was y=−98.51x+10840 (x was the albumin concentration, y was the detection value T), with a coefficient R² of 0.90, and a minimum LOD after dilution was 0.1 ng/mL.

### 4. Screening of a Detection Time of the Immunoassay Device

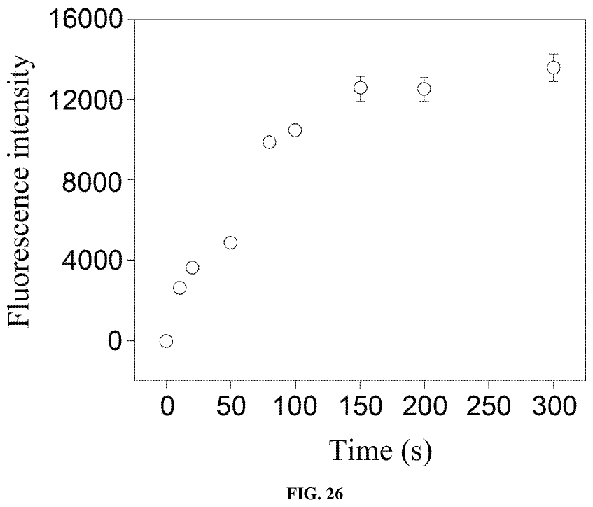

The immunoassay device prepared in Example 1 was used, as shown in FIGS. 5A-B, and the sample with a protein concentration of 50 ng/mL was detected by the detection method in Example 4. The immunoassay analyzer could determine the fluorescent intensity on the test line from 0 s to 300 s. The results were shown in FIG. 26.

As shown in FIG. 26, when the sample was added to the immunoassay device, the detection signal increased to a saturated state within the first 150 s: after a reaction time of 150 s, the intensity of the signal did not change, indicating that the immunoassay device could complete the detection of albumin in the sample within 3 min.

### 5. Specificity Assay of the Immunoassay Device

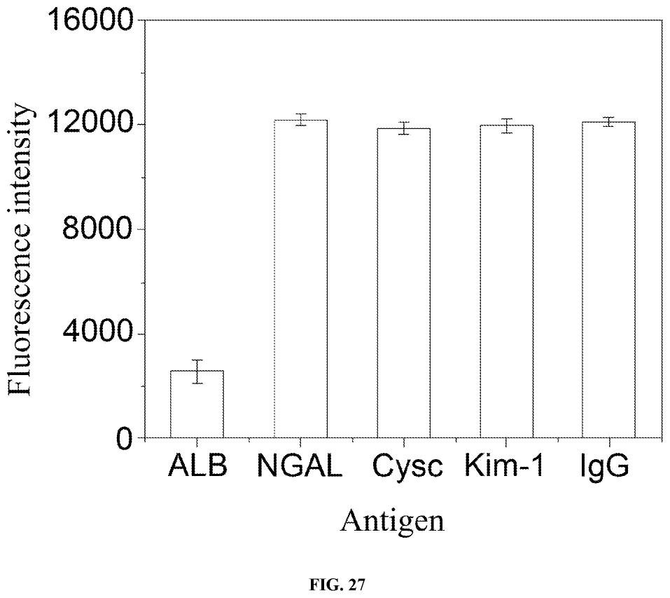

The immunoassay device prepared in Example 1 was used, as shown in FIGS. 5A-B, the samples containing ALB, NGAL, Cysc, Kim-1, and IgG were detected with the detection method in Example 4, where a concentration of each sample was 1 μg/mL. The immunoassay analyzer could determine a fluorescent intensity on the test line. The results were shown in FIG. 27.

The cross-reactivity of LFIA was tested by adding different analytes (ALB, NGAL, Cysc, Kim-1, and IgG) to the test strip. If an analyte other than albumin reacted with the antibody on the conjugate pad, there was no signal in the test line and vice versa. As shown in FIG. 27, only when albumin was added, the test line showed a weak signal: while for other analytes, the fluorescent signal was consistent and strong. The results showed that the immunoassay device had a desirable specificity for the detection of albumin.

### 6. Stability of the Immunoassay Device

The immunoassay device prepared in Example 1 was used, as shown in FIGS. 5A-B, the immunoassay device was stored in a refrigerator at 4° C., and the changes in signal intensity were observed within 30 d.

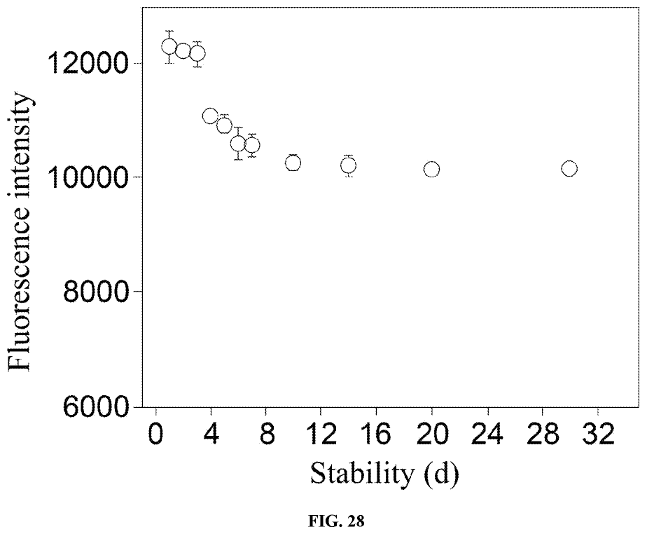

The sample with an albumin concentration of 100 ng/mL was detected by the detection method in Example 4. The immunoassay analyzer could determine a fluorescent intensity on the test line at different days. The results were shown in FIG. 28.

As shown in FIG. 28, during this period, the fluorescent signal decreased by 16.67%, but still maintained a strong signal intensity. This indicated that the test strip showed a desirable stability within 30 d.

### Example 8 Quantitative Analysis of Clinical Sample by Immunoassay Device

The research ethics committee of Peking University First Hospital approved the protocol of this clinical study (approval number: 2022-714).

### 1. Obtaining a Clinical Standard Curve

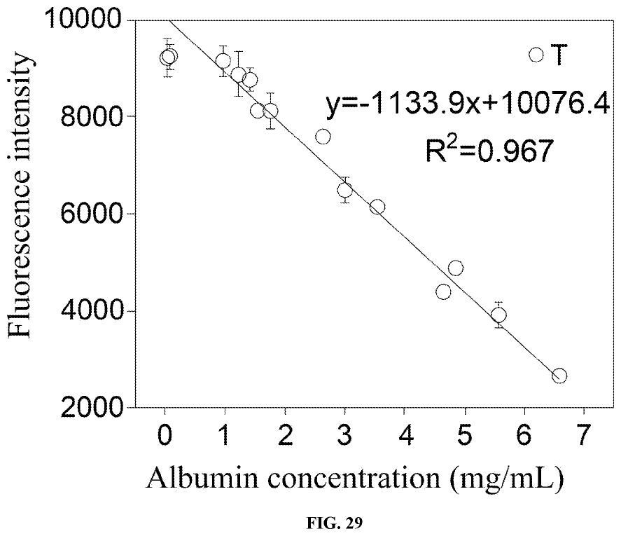

The immunoassay device prepared in Example 1 was used, as shown in FIGS. 5A-B, and 14 clinical urine albumin samples with different concentrations were tested by the detection method in Example 4. The immunoassay analyzer could determine a fluorescent intensity on the test line. The results were shown in FIG. 29.

As shown in FIG. 29, the data points were the test results of the clinical samples using the immunoassay analyzer, and the black line was the fitted standard curve obtained from the clinical samples. The immunoassay device could successfully detect high-concentration clinical samples exceeding 1 mg/mL, a linear equation of the albumin detection was y=−1133.9x+10076.4, with a determination coefficient R² of 0.967. The test results showed that this device had a desirable linear range, the clinical results ranged from (0.1-10) mg/mL, and the LOD after dilution was 0.1 ng/mL.

2. Detection of 14 Clinical Samples with Unknown Concentrations

The immunoassay device prepared in Example 1 was used, as shown in FIGS. 5A-B, and 14 clinical samples with unknown concentrations were tested by the detection method in Example 4. The immunoassay analyzer could determine a fluorescent intensity on the test line. The measured fluorescence intensity was substituted into the standard curve equation in step 1 to obtain a clinical sample concentration of each unknown concentration. At the same time, the above 14 clinical samples with unknown concentrations were measured by the immunoturbidimetric method (Beckman Coulter, AU5800) used by Peking University First Hospital to obtain the corresponding concentration of clinical samples in the hospital.

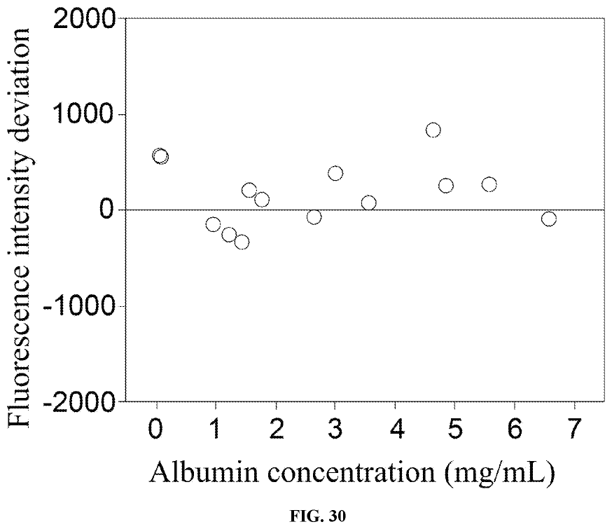

The clinical sample concentration measured by the immunoassay device was compared with the hospital clinical sample concentration to obtain an absolute deviation. The results were shown in FIG. 30 and Table 1.

TABLE 1
Results of the comparison between the concentration of clinical samples measured
by immunoassay devices and the concentration of clinical samples from the hospital

| ALB concentration (mg/mL) | | | |
|---|---|---|---|
| determined by | Fluorescence | ALB concentration | |
| immunoturbidimetric | intensity tested by | (mg/mL) tested by | Deviation |
| method in hospital | immunoassay device | immunoassay device | (%) |
| 0.05 | 9804 | 0.06 | 18 |
| 0.08 | 9795 | 0.07 | −15 |
| 0.97 | 9005 | 0.81 | −17 |
| 1.23 | 8623 | 1.17 | −5 |
| 1.42 | 8453 | 1.33 | −7 |
| 1.56 | 8340 | 1.43 | −8 |
| 1.77 | 8228 | 1.54 | −13 |
| 2.64 | 7541 | 2.18 | −17 |
| 3.01 | 6891 | 2.79 | −7 |
| 3.56 | 6214 | 3.42 | −4 |
| 4.65 | 5219 | 4.36 | −6 |
| 4.85 | 5137 | 4.43 | −9 |
| 5.58 | 4190 | 5.32 | −5 |
| 6.58 | 2574 | 6.84 | 4 |

As shown in FIG. 30 and Table 1, the fluorescence intensity between the concentration of clinical samples measured by the immunoassay device and the concentration of clinical samples from the hospital had an absolute deviation within 500 and a relative deviation within 20%. This indicated that the test results and clinical results were both within confidence limits, and the concentration of albumin in the urine of different patients was successfully measured. Moreover, the test results showed desirable reliability and repeatability. In conclusion, the immunoassay device designed in the present disclosure could successfully realize the dilution and quantitative detection of albumin in urine to detect and diagnose the kidney diseases, and could be used as an auxiliary tool for clinical testing.

The above descriptions are merely preferred implementations of the present disclosure. It should be noted that those of ordinary skill in the art may further make several improvements and modifications without departing from the principle of the present disclosure, but such improvements and modifications should be deemed as falling within the protection scope of the present disclosure.

1. An immunoassay device, comprising a test strip and a thin film pump, wherein a liquid outlet of the thin film pump is closely attached to a top of a conjugate pad of the test strip, or a top of a sample pad of the test strip, or a top of a middle part between the conjugate pad and the sample pad.

2. The immunoassay device according to claim 1, wherein the thin film pump comprises a driving component, a liquid inlet, the liquid outlet, and a solution storage chamber; and an electrode layer in the driving component is connected with a printed circuit board (PCB) or a chip outside the thin film pump through a wire.

3. The immunoassay device according to claim 1, wherein the thin film pump is selected from the group consisting of an electrochemical pump, an electroosmotic pump, and an ultrasonic pump.

4. The immunoassay device according to claim 1, wherein the conjugate pad of the test strip is coated with an antibody-conjugated optical marker; and the optical marker is selected from the group consisting of a fluorescent nanoparticle, a colloidal gold nanoparticle, and an enzyme that catalyzes chemiluminescence.

5. The immunoassay device according to claim 4, wherein an immunoassay method comprises a competitive reaction or a sandwich immunoassay.

6. The immunoassay device according to claim 5, wherein in the competitive reaction, a nitrocellulose membrane of the test strip includes a test line and a control line, the test line is coated with a biomarker of a species to be tested, and the control line is coated with immunoglobulin G (IgG); and
in the sandwich immunoassay, the nitrocellulose membrane of the test strip includes the test line and the control line, the test line is coated with an antibody of the biomarker of the species to be tested, and the control line is coated with the IgG.

7. A preparation method of the immunoassay device according to claim 1, comprising the following steps:
connecting the electrode layer in the driving component in the thin film pump to the PCB or the chip outside the thin film pump through the wire, to control starting or turning off of the thin film pump; wherein the PCB or the chip is attached to a polyvinyl chloride (PVC) backing card on the other side of the test strip;
fixing the nitrocellulose membrane on an upper surface of the PVC backing card, lap-jointing an absorbent pad to a tail end of the PVC backing card, and lap-jointing the sample pad and the conjugate pad sequentially to a head end of the PVC backing card to obtain the test strip; and
attaching the liquid outlet of the thin film pump closely to the top of the conjugate pad of the test strip, or the top of the sample pad of the test strip, or the top of the middle part between the conjugate pad and the sample pad.

8. A preparation method of the immunoassay device according to claim 2, comprising the following steps:
connecting the electrode layer in the driving component in the thin film pump to the PCB or the chip outside the thin film pump through the wire, to control starting or turning off of the thin film pump; wherein the PCB or the chip is attached to a polyvinyl chloride (PVC) backing card on the other side of the test strip;
fixing the nitrocellulose membrane on an upper surface of the PVC backing card, lap-jointing an absorbent pad to a tail end of the PVC backing card, and lap-jointing the sample pad and the conjugate pad sequentially to a head end of the PVC backing card to obtain the test strip; and
attaching the liquid outlet of the thin film pump closely to the top of the conjugate pad of the test strip, or the top of the sample pad of the test strip, or the top of the middle part between the conjugate pad and the sample pad.

9. A preparation method of the immunoassay device according to claim 3, comprising the following steps:
connecting the electrode layer in the driving component in the thin film pump to the PCB or the chip outside the thin film pump through the wire, to control starting or turning off of the thin film pump; wherein the PCB or the chip is attached to a polyvinyl chloride (PVC) backing card on the other side of the test strip;
fixing the nitrocellulose membrane on an upper surface of the PVC backing card, lap-jointing an absorbent pad to a tail end of the PVC backing card, and lap-jointing the sample pad and the conjugate pad sequentially to a head end of the PVC backing card to obtain the test strip; and
attaching the liquid outlet of the thin film pump closely to the top of the conjugate pad of the test strip, or the top of the sample pad of the test strip, or the top of the middle part between the conjugate pad and the sample pad.

10. A preparation method of the immunoassay device according to claim 4, comprising the following steps:
connecting the electrode layer in the driving component in the thin film pump to the PCB or the chip outside the thin film pump through the wire, to control starting or turning off of the thin film pump; wherein the PCB or the chip is attached to a polyvinyl chloride (PVC) backing card on the other side of the test strip;
fixing the nitrocellulose membrane on an upper surface of the PVC backing card, lap-jointing an absorbent pad to a tail end of the PVC backing card, and lap-jointing the sample pad and the conjugate pad sequentially to a head end of the PVC backing card to obtain the test strip; and
attaching the liquid outlet of the thin film pump closely to the top of the conjugate pad of the test strip, or the top of the sample pad of the test strip, or the top of the middle part between the conjugate pad and the sample pad.

11. A preparation method of the immunoassay device according to claim 5, comprising the following steps:
connecting the electrode layer in the driving component in the thin film pump to the PCB or the chip outside the thin film pump through the wire, to control starting or turning off of the thin film pump; wherein the PCB or the chip is attached to a polyvinyl chloride (PVC) backing card on the other side of the test strip;
fixing the nitrocellulose membrane on an upper surface of the PVC backing card, lap-jointing an absorbent pad to a tail end of the PVC backing card, and lap-jointing the sample pad and the conjugate pad sequentially to a head end of the PVC backing card to obtain the test strip; and
attaching the liquid outlet of the thin film pump closely to the top of the conjugate pad of the test strip, or the top of the sample pad of the test strip, or the top of the middle part between the conjugate pad and the sample pad.

12. A preparation method of the immunoassay device according to claim 6, comprising the following steps:
connecting the electrode layer in the driving component in the thin film pump to the PCB or the chip outside the thin film pump through the wire, to control starting or turning off of the thin film pump; wherein the PCB or the chip is attached to a polyvinyl chloride (PVC) backing card on the other side of the test strip;
fixing the nitrocellulose membrane on an upper surface of the PVC backing card, lap-jointing an absorbent pad to a tail end of the PVC backing card, and lap-jointing the sample pad and the conjugate pad sequentially to a head end of the PVC backing card to obtain the test strip; and
attaching the liquid outlet of the thin film pump closely to the top of the conjugate pad of the test strip, or the top of the sample pad of the test strip, or the top of the middle part between the conjugate pad and the sample pad.

13. The preparation method of the immunoassay device according to claim 7, wherein the immunoassay device is placed in a 3D printed shell of a corresponding style; and
the 3D printed shell is prepared by 3D printing or injection molding.

14. The preparation method of the immunoassay device according to claim 8, wherein the immunoassay device is placed in a 3D printed shell of a corresponding style; and
the 3D printed shell is prepared by 3D printing or injection molding.

15. The preparation method of the immunoassay device according to claim 9, wherein the immunoassay device is placed in a 3D printed shell of a corresponding style; and
the 3D printed shell is prepared by 3D printing or injection molding.

16. The preparation method of the immunoassay device according to claim 10, wherein the immunoassay device is placed in a 3D printed shell of a corresponding style; and
the 3D printed shell is prepared by 3D printing or injection molding.

17. The preparation method of the immunoassay device according to claim 11, wherein the immunoassay device is placed in a 3D printed shell of a corresponding style; and
the 3D printed shell is prepared by 3D printing or injection molding.

18. The preparation method of the immunoassay device according to claim 12, wherein the immunoassay device is placed in a 3D printed shell of a corresponding style; and
the 3D printed shell is prepared by 3D printing or injection molding.

19. A method of using an immunoassay device, comprising the following steps: adding a sample to the thin film pump of the immunoassay device according to claim 1, diluting the sample with a diluent in the thin film pump, starting the thin film pump to release a mixed solution of the sample and the diluent, such that the mixed solution flows to the conjugate pad and the nitrocellulose membrane in sequence, and observing or determining a color or an intensity of an optical signal on the test line and the control line in the immunoassay device; alternatively, adding the sample to the sample pad of the said immunoassay device, starting the thin film pump to release the diluent, such that the sample and the diluent are mixed on the test strip to obtain the mixed solution and the mixed solution flows to the conjugate and the nitrocellulose membrane in sequence, and observing or determining the color or the intensity of the optical signal on the test line and the control line in the immunoassay device.

20. The method according to claim 19, wherein the sample is diluted with the diluent by a factor of 1/10 to 1/1000000.
